(19) United States
(12) Patent Application Publication
(10) Pub. No.: US 2016/0051272 A1
(43) Pub. Date: Feb. 25, 2016

(54) ERGONOMIC LOCKING MECHANISM

(71) Applicant: AESCULAP AG, Tuttlingen (DE)

(72) Inventor: Martin STORZ, Tuttlingen (DE)

(21) Appl. No.: 14/780,483

(22) PCT Filed: Jul. 22, 2013
(86) PCT No.: PCT/EP2013/065407

§ 371 Date: Sep. 25, 2015

(30) Foreign Application Priority Data
Apr. 20, 2013 (DE) 10 2013 006 918.1

(51) Int. Cl. A61B 17/28; A61B 17/29

(52) U.S. Cl. CPC A61B 17/2833; A61B 17/2909; A61B 2017/0042

(57) ABSTRACT

The disclosed subjects matter relates to a surgical instrument handle for a surgical instrument, that includes a first handle element, which can be rigidly connected to one of a shaft component and an actuating element of a surgical instrument, wherein an index-finger rest and a ball-of-the-thumb rest are formed on the first handle element, and a second handle element, which is articulated to the first handle element and can be coupled to the other of the shaft component and the actuating element of the surgical instrument, wherein at least one further finger rest is formed on the second handle element. A locking element is rotatably attached to the first handle element, wherein the locking element has an actuation segment and a first locking member. A second locking member is provided on the second handle element, and the first locking member is elastically preloaded toward the second locking member.

The present invention relates to a surgical instrument handle with an ergonomic locking mechanism and in particular to such an instrument handle for tubular shaft instruments.

Surgical instruments have, depending on handle type, a multitude of different locking mechanisms whose function, in case of hemostats, for example, is to take over the holding of the vessel in the closed position so that the user can let go of the instrument without forfeiting the locking effect. On the other hand, the user can at any time release the locking mechanism, remove the instrument and use it again at a different location. Instruments with locking mechanisms are used both in open and in endoscopic surgeries.

In case of ring branch instruments in cross-lever construction, which is the most common instrument construction type in open surgery, toothed bars are arranged on both sides between the branches that engage in the direction of closing and that hook into each other and thus cause a locking effect in the direction of opening. The locking effect is released by tilting the two branches whereby the engagement is lifted. In this process, it is disadvantageous that in releasing, the locking pressure must be overcome whereby the fingers located in the rings must be used against the pressure of the edges of the rings.

In case of shaft instruments, toothed bars that can be swiveled out and are attached at the end of the branches belong to the prior art. Under slight spring pressure, the toothed bar slides over a fixed barbed hook at the opposite branch end so that a locking effect in the direction of opening is created which can be released by swiveling out the toothed bar.

An ergonomic locking mechanism on a surgical instrument handle is in particular important for tubular shaft instruments, since any unergonomic actuation of a locking mechanism results in an undesired movement of the instrument. Due to the relative large distance between the handle and the jaw part of a tubular shaft instrument, a minor undesired twist of the handle part has a significant effect on the jaw part and results in a considerable movement of the same.

In the prior art, numerous instrument handles and locking mechanisms are known. Even instruments with so-called pistol handles are sufficiently known. Pistol handles are handles where the fingers of the hand of the user are essentially bent in the same way as when holding a pistol and the tubular shaft extends above the index finger in approximately the direction in which the extended index finger points. In addition, the thumb and/or the ball of the thumb lies against a first handle element and the index, middle, ring and little fingers lie against a second handle element. One of the two handle elements is rigidly connected with the tubular shaft and the other handle element is articulated to the tubular shaft and also connected to an actuating element. When the two handle elements are moved toward one another, a relative movement of the tubular shaft and the actuating element results which is converted in the jaw part into an opening or closing process. In this way, the jaw part can be opened or closed or held in one position with the pistol handle. So that the user does not have to hold a desired position for a long time, for example when a piece of tissue has to be grasped and held, locking mechanisms are often provided on the pistol handles. The latter create a releasable connection between the two handle elements and in the process ensure their mutual position and thus the position of the jaw part.

In DE 103 53 605 A1, such a pistol handle with a locking mechanism is shown. The locking mechanism can be actuated, i.e. released, using the index finger, by moving the index finger toward the front handle element and actuating a locking element in the process. The locking element has an actuation segment and a locking member, whereby the locking element is pivoted between the actuation segment and the locking member on a projection which extends from the front section of the back handle element downward. A leaf spring thereby pushes the actuation segment proximally from the front handle element, i.e. toward the jaw part. On the locking member, which has a bent design, an arc-shaped toothed segment is provided, the center of which is the articulated connection between the two handle elements and which interacts with a locking tooth that is rigidly formed on the front handle element.

The surgical instrument handle with locking mechanism described above has the following two disadvantages. On the one hand, in order to release the locking effect of the locking mechanism, the index finger must be moved toward the front handle element and must push the actuation segment against the force of the leaf spring toward the front handle element, i.e. distally. At the same time, the front handle element must be moved proximally. Thus, the index finger must perform a movement that is contrary to the movement of the ring, middle and little fingers. This is not a natural movement and therefore unergonomic and easily results in an undesired movement of the instrument. When the index finger pushes the actuation segment of the locking mechanism proximally, it also lies against the index-finger rest, which is slotted and in which the actuation segment is arranged. In this case, the instrument is held securely by the index finger and the thumb or the ball of the thumb. At the moment when the locking mechanism is engaged by moving the index finger away from the actuation segment and thus also from the index-finger rest, the instrument is no longer held securely since now only the thumb or the ball of the thumb and the ring, middle and little fingers effect the holding and the three fingers mentioned last only lie against the actuated front handle element. In this state, an undesired movement of the instrument can easily be caused.

It is therefore the task of the present invention to create a surgical instrument handle that can be held securely at all times and that allows an ergonomic activating or releasing of the locking mechanism. Another task of the present invention is to provide such a locking mechanism where during an opening and closing process, no ratcheting occurs, i.e. where the locking tooth does not slide across the toothed segment.

The task of the present invention is solved by a surgical instrument handle according to claim 1 and a surgical instrument with such a handle according to claim 9. Advantageous embodiments and further developments are the subject matter of the dependent claims.

A surgical instrument handle according to one aspect of the present invention, which is used on a surgical instrument, has the following: a first handle element which can be rigidly connected to one of a shaft component and an actuating element of a surgical instrument, whereby an index-finger rest and a ball-of-the-thumb rest are formed on the first handle element. In addition, it has a second handle element which is articulated to the first handle element and can be coupled to the other of the shaft component and the actuating element of the surgical instrument, wherein at least one further finger rest is formed on the second handle element. In addition, a locking element is rotatably attached to the first handle element, wherein the locking element has an actuation segment and a first locking member. A second locking member is provided on the second handle element, and the first locking member is elastically preloaded toward the second locking member. The first and the second locking members are adapted to interact in order to prevent an increase in the distance between the ball-of-the-thumb rest on the first handle element and the at least one further finger rest on the second handle element. In addition, the actuation segment has a thumb rest, which is adapted to support the thumb of a user substantially from below.

With such a surgical instrument handle, excellent ergonomic handling of the lock is possible. At the same time, the instrument can be held very well and can therefore be precisely positioned and controlled. This is achieved in particular in that the first handle element is held with the index finger and the ball of the thumb. These two extremities have a relatively large distance to one another on a hand so that the handle lever is significantly larger than in other handle designs. In this way, the handle can be held more firmly and significantly less undesired movements of the instrument occur in the hand.

In addition, operation of the locking mechanism is possible by moving the thumb of the hand of the user, with which he holds the handle, substantially downward or upward. This movement is thus neither opposed to any direction of movement of another finger of the holding hand nor interacts with any other movement of the hand or the fingers of the user. When the thumb rest is pushed downward, the first locking member moves out of the second locking member and the instrument handle can be opened slightly. When the thumb rest is pushed upward—whether by the thumb itself or by an elastic element which pushes the thumb rest into its initial position—the second locking member moves into the first locking member and prevents opening of the instrument handle.

According to one advantageous embodiment of the first aspect of the present invention, the locking element is rotatably attached on the first handle element between the actuation segment and the first locking member. With this embodiment, an especially ergonomic arrangement of the actuation segment can be achieved without the locking members having to be arranged too closely to each other at the pivotal point of the two handle elements. As an alternative, the pivotal and mounting point, the actuation segment together with the thumb rest and the first locking member can be formed in one line. In this case, however, the thumb of the user could come into contact with the second locking member, which would be perceived by the user at least as unpleasant and which also constitutes a real hindrance of the opening or closing process of the handle. In addition, the movement of the thumb would have to be controlled very precisely since this construction results in an amplification (or increase) of the movement of the first locking member by means of the different lever lengths.

According to yet another advantageous embodiment of the first aspect of the present invention, an elastic element is arranged between the first handle element and the second handle element, which is adapted to push the handle elements into an open position. Here, the elastic element is preferably a leaf spring that more preferably is attached to the first handle element and lies against the second handle element. With this construction, an opening of the handle does not have to be caused actively by the middle, ring and little fingers. Rather, the handle opens automatically with decreasing locking force which is exerted by the aforementioned fingers. For an actively operated opening of the handle by the fingers referred to, at least one lay-on surface for at least one of the fingers would be required. In this case, at least one of the lay-on surfaces for at least one of the fingers could substantially be designed in the shape of a ring, or a projection is formed between two fingers which forms such a lay-on surface.

According to another advantageous embodiment of the first aspect of the present invention, the index-finger rest, the ball-of-the-thumb rest and/or at least one further finger rest is essentially formed as a closed ring. With such an embodiment, the handle can on the one hand also be opened when no spring pushes the two handle elements into the direction of opening, and on the other hand this type of finger rest forms a kind of guard bracket for the fingers.

According to yet another advantageous embodiment of the first aspect of the present invention, the index-finger rest and the ball-of-the-thumb rest are connected to a bar and the thumb rest is at least in the initial position arranged above the bar. With such an embodiment, a particularly slim and light instrument handle is produced. In addition, there is thus sufficient room on the instrument handle to arrange the thumb rest in a particularly ergonomic manner and to at the same time optimally adapt the locking members to one another and to place them on the handle in such a way that they are not in the way during actuation.

According to a different advantageous embodiment of the first aspect of the present invention, the first locking member has a locking tooth that protrudes towards the second locking member, and the second locking member has a toothed bar that is facing the first locking member and that is preferably designed in the shape of an arc. In this way, the locking tooth can definitely engage with the toothed bar. The preferred arc shape of the toothed bar results in the movement of the locking element for inserting or removing the locking tooth into or out of the toothed bar being identical to one another in any closed position of the handle elements. The closed position taken by the instrument is not always identical and depends in grasping forceps for example on the grasped tissue or element or its thickness. If the required movement of the locking element, and in the process, especially of the actuation segment with the thumb rest, is always identical in any of the possible closed position, this results in a particularly secure handling of the instrument handle.

According to yet another advantageous embodiment of the first aspect of the present invention, the first locking member is preloaded toward the second locking member by means of a leaf spring, which is attached to the first handle element and lies against the locking element.

According to yet another advantageous embodiment of the first aspect of the present invention, the thumb rest extends on both sides of the longitudinal axis of the first handle element 10 so that it can be actuated right-handedly and left-handedly. In this way, no special instrument handle for right handers and left handers must be provided.

According to a second aspect of the present invention, a surgical instrument is disclosed with a shaft component, an actuating element, and a jaw part, which can be actuated by means of an axial relative movement of the shaft component and the actuating element. Here, the surgical instrument has a surgical instrument handle according to one of the preceding claims. With such a surgical instrument, wherein the surgical instrument handle described above is rigidly or removably installed, the advantages described above can be achieved.

According to an advantageous embodiment of the second aspect of the present invention, the first handle element is rigidly connected to one of a shaft component and an actuating element of a surgical instrument and the second handle element is coupled with the other of the shaft component and the actuating element of the surgical instrument. In this way, an instrument is created that can be handled particularly precisely since a rigid connection is often more rigid than a removable connection.

Further advantages and characteristics of the invention are apparent to the person skilled in the art from the attached figures and the detailed description of the exemplary embodiments.

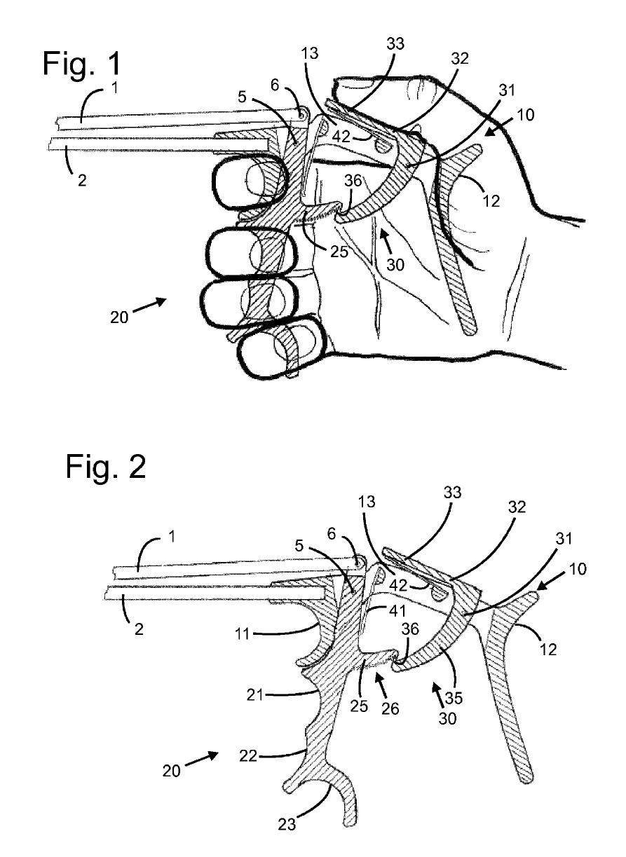

FIG. 1 shows a lateral view of a surgical instrument handle according to a first exemplary embodiment in a hand of a user;

FIG. 2 shows a view corresponding to FIG. 1 without the hand of a user;

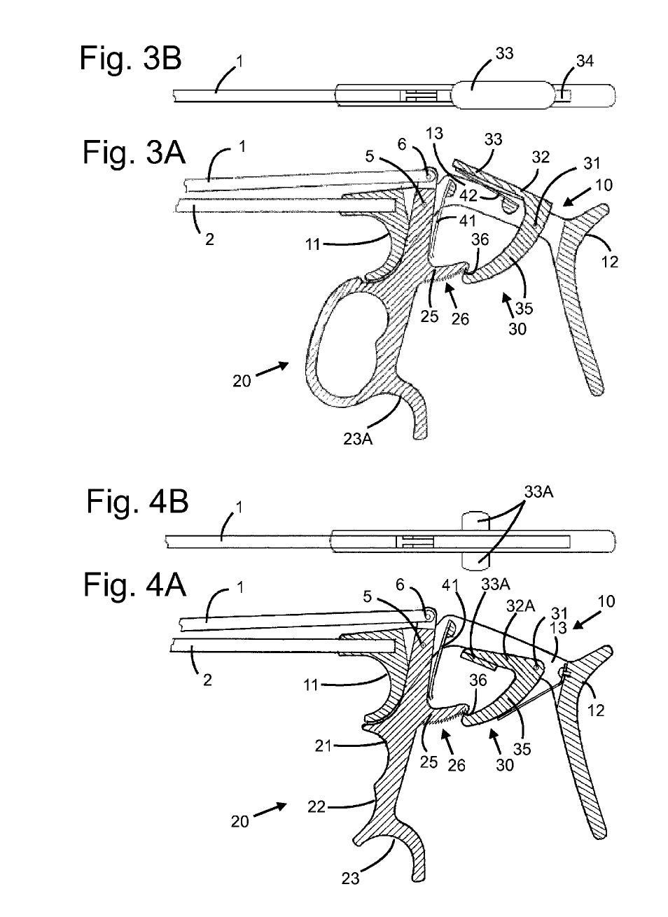

FIG. 3A shows a lateral view of a surgical instrument handle according to a second exemplary embodiment;

FIG. 3B shows a view of the surgical instrument handle according to FIG. 3A from above;

FIG. 4A shows a lateral view of a surgical instrument handle according to a third exemplary embodiment; and

FIG. 4B shows a view of the surgical instrument handle according to FIG. 4A from above;

A first exemplary embodiment of the present invention is described in detail with reference to FIGS. 1 and 2.

The surgical instrument handle of this exemplary embodiment is created for a surgical instrument not described in more detail, such as endoscopic grasping forceps. Shaft component 1 and actuating element 2 are only shown schematically and the actuating element 2 can indeed run in a shaft component 1. In order to prevent an upward or downward movement of the actuating element 2 during the actuation of the instrument handle, the actuating element can be coupled with a pin to an elongated hole that is formed in the second handle element in place of the articulated joint 6.

The instrument handle has a first handle element 10 which is rigidly connected to the shaft component 1. On the first handle element 10, an index-finger rest 11 and a ball-of-the-thumb rest 12 are formed into which the index finger and the ball of the thumb of the user are inserted respectively. The two rests 11 and 12 are connected by means of a bar 13. A second handle element 20 which is connected to the first handle element 10 at a point of articulation 5 is coupled to the articulated joint 6 by the actuating element 2 of the surgical instrument. On the second handle element 20, three individual finger rests 21, 22, 23 are formed. To the first handle element 10, a locking element 30 is rotatably attached, which has an actuation segment 32 and a first locking member 35. The first locking member is a single locking tooth 36. On the second handle element 20, a second locking member 25 is provided in the form of a toothed bar 26 whose side facing the locking tooth 36 is arc-shaped. The first locking member 35 is elastically preloaded toward the second locking member 25 by means of a leaf spring 42. The leaf spring 42 is attached to the bar 13 of the first handle element 10 and essentially lies from below against a thumb rest 33 which is formed on the actuation segment 32 of the locking element 30.

The locking tooth 36 and the toothed bar 26 are adapted to interact in order to prevent an increase in the distance between the ball-of-the-thumb rest 12 on the first handle element 10 and the finger rests 21, 22, 23 on the second handle element 20. For this purpose, the locking tooth 36 can enter the spaces between the teeth of the toothed bar 26. The form of the locking tooth 36 and of the teeth of the toothed bar 26 makes it possible that the locking tooth 36 can slide across the toothed bar 26 in the direction of closing without the locking element 30 being or having to be actuated, whereas a movement in the opposite direction without actuating the locking element 30 is safely prevented. Corresponding forms of the toothed bar 26 and the locking tooth 36 are known in the prior art. The thumb rest 33 is designed in such a way that it essentially supports the thumb of a user from below as shown in FIG. 1. In addition, the thumb rest 33 in this exemplary embodiment is always located above the bar 13 and extends laterally on both sides of the bar 13 so that the instrument handle can be used by right handers and left handers.

The point 31 where the locking element 30 is articulated on the bar 13 of the first handle element 10 is located between the actuation segment 32 with the thumb rest 33 and the first locking member 35 with the locking tooth 36. A leaf spring 41 is arranged between the first handle element 10 and the second handle element 20 and pushes the handle elements 10, 20 into an open position. The leaf spring 41 is attached to the first handle element 10 and lies against the second handle element 20.

By slightly bending the thumb of a user, the thumb tip, which is located on the thumb rest 33 in an ergonomic hand position, moves downward. In the process, the locking element 30 is pivoted about the point of articulation 31 and the locking tooth 36 of the first locking member 35 moves out of engagement with the teeth of the toothed bar 26 of the second locking member 25 which is formed as one piece with the second handle element 20 and protrudes toward the first handle element 10. In this state, any desired opening and closing of the instrument handle and thus of the associated jaws of the instrument can be effected. In order to lock a closed position, i.e. in order to prevent an unwanted opening from this position, the user extends the thumb to the point where the thumb rest 33 and thus the locking member [sic element] 30 returns to its initial position. In doing so, the locking tooth 36 engages with the toothed bar 26 and the instrument handle is locked.

A second exemplary embodiment of the present invention is described below with reference to FIG. 3A and 3B whereby mainly the differences to the first exemplary embodiment are described here. The only differences to the first exemplary embodiment are the thumb rest 33 being constructed relatively small and the finger rests for the middle finger and the ring finger being formed as a common ring-shaped rest 21A. As shown in FIG. 3B, the bar 13 of the first handle element 10 is slotted and the second handle element 20 as well as the locking element 30 are arranged in the slot 34. In this way, the first handle element 10 has effectively 2 bars. In principle, the first handle element 10 can also have only one bar 13 and the second handle element 20 as well as the locking member [sic element] 30 are not arranged in the slot 34 but at the side of the then only bar 13.

A third exemplary embodiment of the present invention is described in detail below with reference to FIG. 4A and 4B whereby again mainly the differences to the first exemplary embodiment are described. The locking element 30A has a very wide thumb rest 33B, which is additionally arranged below the bar 13. The spring 42A, with which the locking element 30A is pushed into the locked position, is attached in the area of the ball-of-the-thumb rest 12 and lies diagonally against the bottom of the first locking member 35. The ball-of-the-thumb rest [sic thumb rest] 33B is therefore designed so wide that the thumb in the position for use is now located not above the bar 13 but next to the bar 13. In order to prevent an accidental sliding of the thumb off the thumb rest 33B, the latter has to be dimensioned accordingly.

For the specialist, multiple additional embodiments and modifications of the present invention also arise from the attached claims, the above description and the figures. Of course, individual features or groups of features of the different exemplary embodiments can be combined with one another in any suitable manner. The leaf springs can, for example, also be substituted by leg springs or torsion springs.

1. A surgical instrument handle for a surgical instrument with
a first handle element which can be rigidly connected to one of a shaft component and an actuating element of a surgical instrument, wherein an index-finger rest and a ball-of-the-thumb rest are formed on the first handle element,
a second handle element, which is articulated to the first handle element and can be coupled to the other of the shaft component and the actuating element of the surgical instrument,
wherein at least one further finger rest is formed on the second handle element,
wherein
a locking element is rotatably attached to the first handle element, wherein the locking element has an actuation segment and a first locking member,
a second locking member is provided on the second handle element,
the first locking member is elastically preloaded toward the second locking member,
the first and the second locking members are adapted to interact in order to prevent an increase of the distance between the ball-of-the-thumb rest on the first handle element and the at least one further finger rest on the second handle element, and
the actuation segment has a thumb rest which is adapted to support the thumb of a user substantially from below.

2. The surgical instrument handle according to claim 1, wherein the locking element is rotatably attached on the first handle element between the actuation segment and the first locking member.

3. The surgical instrument according to claim 1, wherein
an elastic element is arranged between the first handle element and the second handle element, which is adapted to push the handle elements into an open position, wherein the elastic element is preferably a leaf spring which more preferably is attached to the first handle element and lies against the second handle element.

4. The surgical instrument according to claim 1, wherein
the index-finger rest, the ball-of-the-thumb rest and/or at least one further finger rest are formed as an essentially closed ring.

5. The surgical instrument according to claim 1, wherein
the index-finger rest and the ball-of-the-thumb rest are connected to a bar and the thumb rest is arranged at least in the initial position above the bar.

6. The surgical instrument according to claim 1, wherein
the first locking member has a locking tooth that protrudes toward the second locking member, and
the second locking member has a toothed bar that faces the first locking member and is preferably designed in the shape of an arc.

7. The surgical instrument according to claim 1, wherein
the first locking member is preloaded toward the second locking member by means of a leaf spring which is preferably attached to the first handle element and lies against the locking element.

8. The surgical instrument according to claim 1, wherein the thumb rest extends on both sides of the longitudinal axis of the first handle element so that it can be actuated right-handedly and left-handedly.

9. A surgical instrument, comprising:
a shaft component,
an actuating element,
a jaw part which can be actuated by means of an axial relative movement of the shaft component and the actuating element, wherein
a surgical instrument handle is made according to claim 1.

10. The surgical instrument according to claim 9, wherein
the first handle element is rigidly connected to one of a shaft component and an actuating element of a surgical instrument, and
the second handle element is coupled to the other of the shaft component and the actuating element of a surgical instrument.
